(19) United States
(12) Patent Application Publication
(10) Pub. No.: US 2011/0160560 A1
(43) Pub. Date: Jun. 30, 2011

(54) Pressure sensor apparatus, system and method

(76) Inventor: Robert T. Stone, Sunnyvale, CA (US)

(21) Appl. No.: 12/655,405

(22) Filed: Dec. 29, 2009

(51) Int. Cl. A61B 3/16; G01L 19/04; G01L 9/12; A61B 5/00

(52) U.S. Cl. 600/398; 73/708; 73/724; 600/561

(57) ABSTRACT

An implantable pressure sensor system having a sensor assembly configured and adapted to measure pressure in a volume, the sensor assembly including at least a first MEMS pressure sensor, an application-specific integrated circuit (ASIC) having memory means, temperature compensation system, drift compensation system, and power supply means for powering the sensor assembly, the first MEMS pressure sensor having a pressure sensing element that is responsive to exposed pressure, the pressure sensing element being adapted to generate a pressure sensor signal representative of the exposed pressure, the temperature compensation system being adapted to correct for temperature induced variations in the pressure sensor signal, the drift compensation system being adapted to correct for pressure and temperature induced pressure sensor signal drift.

### FIELD OF THE PRESENT INVENTION

The present invention relates generally to apparatus and methods for measuring pressure within a cavity. More particularly, but not by way of limitation, the invention relates to apparatus, systems and methods for measuring pressure within a cavity of a subject.

### BACKGROUND OF THE INVENTION

In medical diagnosis and treatment of a subject or patient, it is often necessary to measure the pressure within one or more organs or systems in the subject's body. Examples of pertinent pressures include, without limitation, intraocular pressure, intratracheal or respiratory pressure, arterial pressure, and bladder pressure.

As is well known in the art, intraocular pressure is a risk factor for the development and progression of glaucoma and other visual impairment conditions. Reduction of intraocular pressure has been shown to reduce the risk of developing glaucoma, as well as the risk of disease progression.

Various conventional systems and methods have thus been developed to assess intraocular pressure during a clinic visit. A major drawback of the conventional systems and methods is, however, a patient that presents with acceptable intraocular pressure during clinic office hours may experience intraocular peaks at other times during the day. It is the opinion of many in the field that fluctuations in intraocular pressure may be an independent risk factor for several visual diseases.

An increase in intraocular pressure during a nocturnal period, combined with a decrease in blood pressure, which often occurs during a nocturnal period, can also compromise optic nerve head flow in susceptible individuals.

Several intraocular sensor systems and methods have thus been proposed to continuously measure intraocular pressure. Illustrative are the systems and associated methods disclosed in U.S. Pat. Nos. 6,443,893 and 7,481,534. The systems disclosed in the noted patents include an implantable sensor device, a wireless transmitter and an external receiver or reader.

Although the disclosed intraocular pressure sensor systems provide effective means for measuring intraocular pressure, the systems are highly complex and not suitable for long term use.

A further, highly pertinent pressure, which often times needs to be closely monitored, is intracranial pressure. Elevated intracranial pressure can be tolerated for only a few hours or perhaps as long as days or weeks. In all circumstances, unmonitored and uncontrolled elevated intracranial pressure will eventually lead to visual loss or to cerebral white matter injury and dementia.

The general physiological states and processes that can elevate intracranial pressure include brain tumors, pseudotumor cerebri, hydrocephalus, sever head trauma and other situations where subjects or patients present brain swelling, edema, obstruction of cerebral spinal fluid pathways or intracranial space occupying lesions. Accurate monitoring of intracranial pressure in these situations frequently allows correctional emergency procedures when intracranial pressure rises or falls to dangerous levels.

Various conventional apparatus, systems and methods have thus been employed to monitor intracranial pressure. One currently available method for monitoring intracranial pressure comprises measuring cerebral spinal fluid pressure via a lumbar puncture. Another available method comprises directly measuring intracranial pressure using a catheter, which is inserted into and though the scalp and skull. The catheter is connected to an external data acquisition system. In some cases, the catheter is simply a plastic tube that vents the subarachnoid pressure to an electronic readout pressure gauge.

There are several drawbacks associated with the noted conventional systems and methods. The drawbacks include the incumbent risks associated with insertion of medical apparatus, e.g., catheter, tubes, etc., into and through the skull, post-insertion infection, and susceptibility to disruption and dislodgement by the subject and/or hospital personnel.

A more recently developed class of pressure sensors comprises microfabricated or microminiature (MEMS) pressure sensors. MEMS pressure sensors typically measure pressure by detecting the strain induced on a pressure sensing element, i.e. transducer. The sensor converts the strain into an electrical signal by measuring the resistance on the strained element, such as is done in piezoresistive-based sensors. Illustrative are the MEMS pressure sensors disclosed in U.S. Pat. Nos. 7,196,385 and 7,028,550.

There are similarly several drawbacks associated with MEMS pressure sensors and associated methods employing the sensors. A significant drawback is that over extended periods of use, the pressure sensors experience drift.

Drift is the irreversible shift (or distorting changes) to a sensor's base line readings, i.e. initial response curve, over time. Sensor drift can result from various sources and/or mechanisms, which fundamentally alter the chemical or metallurgical properties of the sensor or structures thereof. Such sources include exposure to high pressures and/or high (or fluctuating) temperatures for extended periods of time.

The level of drift can also vary between manufactured lots of sensors due to variations in the chemical and/or metallurgical properties of the materials employed in the sensors.

As is well known in the art, sensor drift adversely affects the accuracy of the sensor output and, hence, the accuracy of physiological parameters determined therefrom. Drift obscures accurate data both by producing false positive and false negative readings. By way of example, false negative results can occur when drift of base-line data distorts or fully obscures a sensor signal representing a physiological parameter change, which would otherwise be indicative of the physiological parameter change. This occurs when the drift moves a “0” base line level into a negative range. Conversely, when sensor drift is in a positive range, a sensor signal can be mistaken for a change in a physiological parameter, running the risk of a false indication of an adverse physiological parameter or condition.

Unfortunately, sensor drift is typically unpredictable. Thus, sensor drift can not be simply factored out via a mathematical algorithm or calculation(s) to compensate for the data distortion.

Drift is particularly problematic with implantable sensors, where recalibration opportunities are limited or impractical. Because of the limited ability to recalibrate implanted sensors, the failure of most currently available pressure sensors to remain stable (i.e. free of drift) has made them unsuitable for long term implantable use.

It would thus be a significant advancement in the art to provide pressure sensors, and associated systems and methods, which provide accurate and stable sensor output under varying conditions and over extended periods of time.

It is therefore an object of the present invention to provide pressure sensors, and associated systems and methods, which provide accurate and stable sensor output under varying in vivo and ambient conditions.

It is another object of the invention to provide pressure sensors, and associated systems and methods, which provide accurate and stable sensor output over extended periods of time.

It is another object of the invention to provide implantable pressure sensors, and associated systems and methods, which provide accurate and stable sensor output under varying in vivo and ambient conditions, and over extended periods of time.

It is another object of the invention to provide implantable pressure sensors and associated systems that are suitable for long term implantable use.

### SUMMARY OF THE INVENTION

In accordance with the above objects and those that will be mentioned and will become apparent below, the pressure sensor system, in accordance with one embodiment of the invention, generally comprises a sensor assembly configured and adapted to measure pressure in a volume, the sensor assembly including at least one MEMS pressure sensor, a temperature compensation system, a drift compensation system, and power supply means for powering the sensor assembly, the MEMS pressure sensor being responsive to exposed pressure and adapted to generate a pressure sensor signal representative of the exposed pressure, the temperature compensation system being adapted to correct for at least one temperature induced variation of the pressure sensor signal, the drift compensation system being adapted to correct for pressure induced pressure sensor signal drift.

In another embodiment of the invention, the pressure sensor system generally comprises a sensor assembly having at least one MEMS pressure sensor, a temperature compensation system, a drift compensation system, and a pressure compensation system, the MEMS pressure sensor being responsive to exposed pressure and adapted to generate a pressure sensor signal representative of the exposed pressure, the temperature compensation system being adapted to correct for temperature induced variations in the pressure sensor signal, the drift compensation system being adapted to correct for pressure and temperature induced drift of the pressure sensor signal, the pressure compensation system being adapted to correct for variations in measured pressures of the MEMS pressure sensor and atmospheric pressure.

In another embodiment of the invention, the pressure sensor system generally comprises a sensor assembly having a MEMS pressure sensor, an application-specific integrated circuit (ASIC), a temperature compensation system, a drift compensation system, and a pressure compensation system, the MEMS pressure sensor having a pressure sensing element that is adapted to generate a capacitance variation signal in response to exposed pressure, the ASIC being adapted to generate a pressure signal with the capacitance variation signal, the pressure signal being representative of the exposed pressure, the temperature compensation system being adapted to correct for temperature induced variations in the pressure signal, the drift compensation system being adapted to correct for pressure and temperature induced drift of the capacitance variation signal, the pressure compensation system being adapted to correct for variations in measured pressures of the MEMS pressure sensor and atmospheric pressure.

In another embodiment of the invention, the pressure sensor system generally comprises a MEMS pressure sensor, a digital capacitance system, a temperature compensation system, a drift compensation system, and a pressure compensation system, the MEMS pressure sensor being adapted to generate a capacitance signal in response to exposed pressure, the digital capacitance system being adapted to convert the capacitance signal to a pressure signal, the pressure signal being representative of the exposed pressure, the temperature compensation system being adapted to correct for temperature induced variations in the capacitance signal, the drift compensation system being adapted to correct for pressure and temperature induced drift of the capacitance signal, the pressure compensation system being adapted to correct for variations in measured pressures of the MEMS pressure sensor and atmospheric pressure.

In accordance with another embodiment of the invention, there is provided a method of measuring pressure in a chamber of a human body comprising the steps of (i) providing a sensor assembly having a MEMS pressure sensor, an application-specific integrated circuit (ASIC), a temperature compensation system, a drift compensation system, a pressure compensation system and power supply means for powering the sensor assembly, the MEMS pressure sensor being adapted to generate a capacitance variation signal in response to exposed pressure, the ASIC being adapted to generate a pressure signal with the capacitance variation signal, the pressure signal being representative of the exposed pressure, the temperature compensation system being adapted to correct for temperature induced variations in the capacitance variation signal, the drift compensation system being adapted to correct for pressure and temperature induced drift of the capacitance variation signal, the pressure compensation system being adapted to correct for variations in measured pressures of the MEMS pressure sensor and atmospheric pressure, (ii) disposing the sensor assembly in a chamber of a human body, and (iii) measuring pressure in the chamber with the sensor assembly, whereby a first pressure signal representative of the chamber pressure is generated.

In some embodiments of the invention, the chamber comprises an anterior chamber of an eye.

In some embodiments of the invention, the chamber comprises an intracranial chamber.

In some embodiments of the invention, the sensor assembly includes wireless communication means for wirelessly transmitting the first pressure signal to a remote receiving apparatus.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a partial cross-sectional, front plane view of one embodiment of a pressure sensor, according to the invention;

FIG. 2 is a cross-sectional, front plane view of one embodiment of a MEMS pressure sensor, according to the invention;

FIG. 3 is a schematic illustration of one embodiment of an ASIC module, according to the invention;

FIG. 4A is a schematic illustration of one embodiment of a telemetric pressure sensor system, according to the invention;

FIG. 4B is a top plane view of the system reader shown in FIG. 4A, according to the invention;

FIG. 5 is a schematic illustration of one embodiment of a wired pressure sensor system, according to the invention;

FIG. 6 is a partial sectional, front plane view of one embodiment of an intracranial pressure (ICP) sensor, according to the invention;

FIG. 7 is a top plane view of one embodiment of an ICP sensor housing, according to the invention;

FIG. 8 is an illustration of a subject's head showing the pre-placement positioning of the ICP sensor shown in FIG. 6, according to one embodiment of the invention;

FIG. 9 is an illustration of one embodiment an ICP sensor system, wherein the ICP sensor shown in FIG. 6 is implanted in the skull of a subject and an external reader is disposed external to the ICP sensor, according to the invention;

FIG. 10 is an illustration of one embodiment an intraocular pressure sensor positioned on the eye of a subject, according to the invention; and

FIG. 11 is a front plane view of the intraocular pressure sensor shown in

FIG. 10, according to the invention.

### DETAILED DESCRIPTION OF THE INVENTION

Before describing the present invention in detail, it is to be understood that this invention is not limited to particularly exemplified apparatus, systems, materials, structures or methods, as such may, of course, vary. Thus, although a number of apparatus, systems, materials, structures and methods similar or equivalent to those described herein can be used in the practice of the present invention, the preferred apparatus, systems, materials, structures and methods are described herein.

It is also to be understood that the invention is not limited to any particular application used herein in connection with a described embodiment of the invention.

Further, the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Where a range of values is provided, it is to be understood that each intervening value, to the tenth of a unit of the lower limit unless the context clearly dictates otherwise, between the upper and lower limit of that range and any other stated or intervening value in that stated range, falls within the scope of the invention. The upper and lower limits of these smaller ranges may independently be included in the smaller ranges and also fall within the scope of the invention, subject to any specifically excluded limit in the stated range. Where the stated range includes one or both of the limits, ranges excluding either or both of those included limits also fall within the scope of the invention.

As used in this specification and the appended claims, the singular forms “a”, “an” and “the” include plural referents unless the content clearly dictates otherwise. As such, this statement is intended to serve as antecedent basis for use of such exclusive terminology as “solely”, “only” and the like in connection with the recitation of claim elements, or use of a “negative” limitation.

Further, all publications, patents and patent applications cited herein, whether supra or infra, are hereby incorporated by reference in their entirety.

The publications, patents and published patent applications discussed herein are provided solely for their disclosure prior to the filing date of the present application. Nothing herein is to be construed as an admission that the present invention is not entitled to antedate such publications, patents and published patent applications by virtue of prior invention.

### DEFINITIONS

The term “a volume”, as used herein, means any space, chamber, cavity, substance, tissue, area or the like.

The term “physiologic”, as used herein, means that in certain embodiments of the invention, the pressure sensors (and associated systems), which are described in detail below, are configured (e.g., shaped, dimensioned, etc.) so that they can be positioned in or on a body of a living organism, e.g., a human.

The terms “patient” and “subject”, as used herein, mean and include humans and animals.

As summarized above, in certain embodiments, the present invention comprises improved pressure sensors, pressure sensor systems, and methods for their preparation and use. In further describing the subject invention, the subject sensors, sensor systems and their preparation are described first in greater detail, followed by a review of representative methods in which they find use.

Several embodiments of the pressure sensors and associated systems of the invention will now be described in detail. For simplicity and without limitation, subheadings are used to organize the descriptions.

### Pressure Sensor Configurations

The pressure sensors of the invention are designed and adapted to accurately measure pressure in a volume. As indicated above, “a volume”, as used herein, means any space, chamber, cavity, substance, tissue, area or the like. In connection with the pressure sensor embodiments, and associated systems and methods described herein, a volume comprises a chamber of a human body, such as a cranial cavity, but this is only one example of a volume, and the invention is in no way limited to such a chamber. According to the invention, “a volume” can also comprise a space, chamber, cavity or the like that is not in a human body. The pressure sensors of the invention can also be employed in a wide variety of non-medical contexts. Therefore, although the following discussion generally focuses on measuring pressure in cavities and chambers in a human body, the invention is in no way limited to such application.

As will readily be appreciated by one having ordinary skill in the art, the pressure sensors, and associated systems and methods of invention, provide several significant advantages compared to prior art pressure sensors and methods. A significant feature and, hence, advantage of the pressure sensors of the invention is that they provide very accurate and stable outputs over extended periods of time. The pressure sensors can thus be positioned in (or on) a body for extended periods of time, e.g., months or even years, without significant, if any, functional deterioration, i.e. the sensor structures exhibit minimal, if any, drift.

As stated above, drift is the irreversible shift (or distorting changes) to a sensor's base line readings, i.e. initial response curve, over time. Drift can, and in most instances will, adversely affect the accuracy of the sensor output and, hence, the accuracy of physiological parameters determined therefrom.

As is well known in the art, drift rates for a given sensor structure can be determined by monitoring the output of the sensor over a period of time when the sensor is employed in a typical use environment, or model thereof. In such tests, drift can be assessed by maintaining pressure at a stable value, e.g., constant value, and monitoring the output of the sensor over time in order to ascertain if there are any changes or shift in the sensor output. Drift can also be assessed by varying the temperature and similarly monitoring the output of the sensor over time in order to ascertain if there are any changes or shift in the sensor output. The observed changes in the sensor output, if any, can then be employed to determine the sensor drift characteristics.

The drift test that is often employed is one that accelerates the drift process that occurs naturally in an in situ environment, whereby useful data can be acquired without waiting for the full lifetime of a sensor to pass. There are various known methods that can be employed to accelerate the external factors that cause pressure sensor drift.

Whatever drift test is employed, in certain embodiments of the invention, the pressure sensors (and associated systems) will exhibit minimal, if any, drift over a period from approximately 1-10 years or more. Indeed, in some embodiments of the invention, the pressure sensors will exhibit a drift of no more than 1-2 mmHg/year.

The noted low drift characteristic of the subject pressure sensors is in sharp contrast to the drift observed in many current prior art pressure sensors, where the sensor drift can be 7 mmHg/hr or greater.

As summarized above, the pressure sensors of the invention generally include a housing or case, a pressure sensing system, power supply means (or an energy source), and communication means. The pressure sensors also preferably include a temperature or drift compensation system.

In certain embodiments of the invention, the pressure sensor systems of the invention additionally include a pressure compensation system.

In certain embodiments of the invention, the pressure sensing system includes at least a first MEMS pressure sensor, which is disposed in the sensor housing. In certain embodiments, the pressure sensing system includes at least two MEMS pressure sensors; at least one MEMS pressure sensor being disposed in the sensor housing and at least one MEMS pressure sensor being disposed at an external position, e.g., at the end of a coupled cable or an external reader.

In certain embodiments, the MEMS pressure sensors and, hence, pressure sensors associated therewith are adapted to measure pressure changes in a volume with a sensitivity (or accuracy) of at least approx. +/−0.75 mmHg on a scale of approximately 500-1000 mmHg.

In certain embodiments, the MEMS pressure sensors (and systems associated therewith) have an operating temperature in the range of approximately 35-42° C.

In certain embodiments of the invention, the sensor housing is adapted to securely position at least one MEMS pressure sensor, and associated components, modules and circuitry, within the sensor housing. In certain embodiments, the housing is further designed and adapted to facilitate placement of the pressure sensor in or on a subject's body.

In certain embodiments of the invention, the communication means includes a communication network or link. In certain embodiments, the communication link comprises a wireless link, i.e. telemetric pressure sensors. In certain embodiments, the communication link comprises conductive wires or similar direct communication means.

In certain embodiments of the invention, the pressure sensors also include at least one additional sensor, preferably, a MEMS sensor. According to the invention, the additional sensor can include, without limitation, a temperature sensor, pO₂ sensor, pCO₂ sensor, and SpO₂ sensor.

As also summarized above, in certain embodiments, the pressure sensors of the invention employ selected materials (and associated processing means) and a unique component configuration, which impart a low drift characteristic to the pressure sensor structure. In certain embodiments, the pressure sensors of the invention include a unique digital capacitance system and an application-specific integrated circuit (ASIC) that provides translation from capacitance variation to pressure and individual correction for a calibrated temperature coefficient, which also significantly enhance the accuracy of the sensor output(s) when subjected to varying conditions.

Referring now to FIG. 1, there is shown one embodiment of a pressure sensor 10 of the invention. As illustrated in FIG. 1, the pressure sensor 10 generally includes a housing or case 12, a membrane 14 disposed at a first end, and a cap 16, having a lumen or feed-through 18 therethrough, disposed on a second end.

Disposed within the sensor housing 12 is a sensor module (i.e. pressure sensing system) 30, an ASIC module 40, and associated circuitry 21 that facilitates communication by and between the sensor module 30, ASIC module 40 and the communication means.

As illustrated in FIG. 1, also disposed within the sensor housing 12 is a pressure transmitting fluid 20. In certain embodiments, the pressure transmitting fluid 20 comprises silicon oil.

As indicated above, in certain embodiments of the invention, the sensor housing 12 is designed and configured to facilitate placement of the sensor 10 in or on a subject's body. Thus, in certain embodiments, the housing comprises a biocompatible material, such as, without limitation, stainless steel, silicon, titanium and polyetheretherketone (PEEK).

In certain embodiments, the membrane 14 similarly comprises a biocompatible material, such as titanium, stainless steel and silicon. In certain embodiments, the membrane 14 comprises titanium.

As also indicated above, in certain embodiments of the invention, the sensor module 30 includes at least one MEMS pressure sensor. In certain embodiments, the MEMS pressure sensors of the invention comprise absolute pressure sensors that are capacitive and optimized to operate within a range of approximately 700-1300 mbar. The MEMS pressure sensors thus include at least one contact that provides access to a measurement capacitance and, in certain embodiments, a reference capacitance.

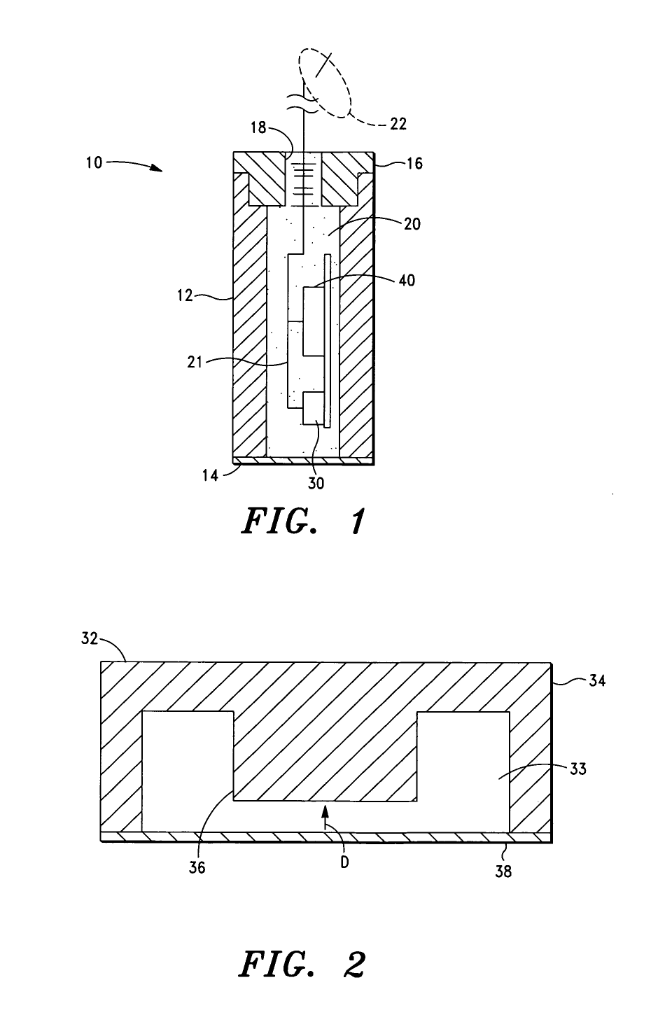

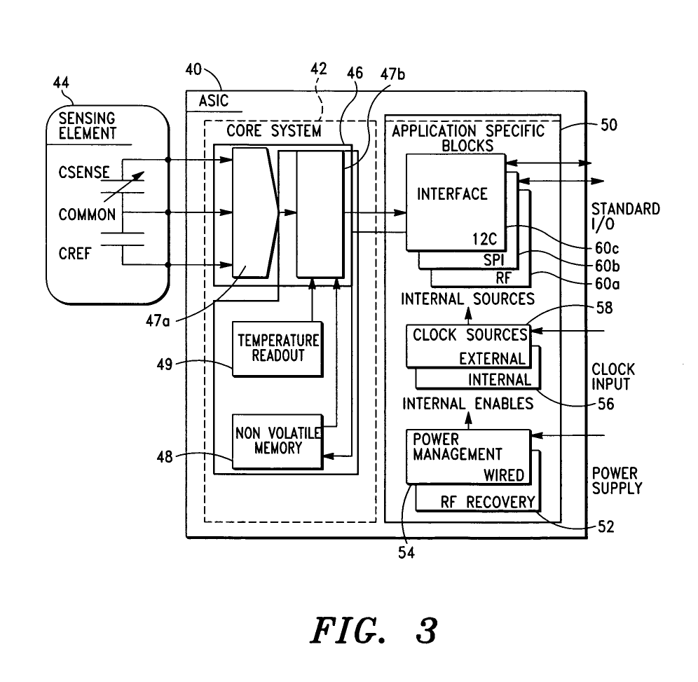

Referring now to FIG. 2, there is shown one embodiment of a MEMS pressure sensor 32 of the invention. As illustrated in FIG. 2, the MEMS pressure sensor 32 includes a housing 34, having an internal cavity 33 and a diaphragm (or sensing element) 38. The housing 34 further includes an internal post 36 that is configured and positioned to limit pressure induced deflection of the diaphragm 38 (as shown by arrow “D”).

In certain embodiments, associated with the sensor system 30 and, hence, MEMS pressure sensor(s) associated therewith, is a digital capacitance system, i.e. a dedicated electronic processing circuit. In certain embodiments, the noted processing comprises at least one digital conversion of the measured sensor signal.

In certain embodiments, the diaphragm 38 comprises monocrystalline silicon. In certain embodiments, the monocrystalline silicon is metallicized.

A key feature of the MEMS pressure sensor 32 of the invention is that it is hermetically sealed. Whereas prior art sensor designs have attempted to incorporate a sensing membrane and moisture resistant seal, the present invention incorporates a totally hermetic seal, e.g. titanium metal and ceramic feedthrough, to totally prevent moisture (even in a monomolecular state) from diffusing through the membrane or housing and ultimately degrading the performance of the MEMS pressure sensor.

Referring back to FIG. 1, in certain embodiments of the invention, the ASIC module 40 is in communication and, hence, associated with the sensor module 30, sensor digital capacitance system, and communication means. In certain embodiments, the ASIC module 40 is designed and adapted to perform at least one of the following functions: (i) compare a variable current to a reference capacitance, (ii) provide signal shaping, (iii) provide pressure sensor signal correction based on calibrated temperature coefficient, (iv) provide power management, (v) provide communications to external circuitry, and (vi) control signal transmissions to/from the pressure sensor 10.

Referring now to FIG. 3, there is shown a schematic illustration of one embodiment of an ASIC module 40 of the invention. As illustrated in FIG. 2, the ASIC module 40 generally includes a core system 42, an application specific subsystem 50, and a temperature sensing element 44, which preferably is in communication with the core system 42.

In the illustrated embodiment, the core system 42 includes first processing means 46 that is in communication with the temperature sensing element 44. As illustrated in FIG. 3, in one embodiment of the invention, the first processing means 46 includes at least two first processing means modules 47a, 47b. In certain embodiments, the first module 47a is preferably adapted to provide or effectuate current conversion (i.e. AC conversion C/D). In certain embodiments, the second module 47b is preferably adapted to provide signal shaping.

As further illustrated in FIG. 3, in certain embodiments, the core system 42 further includes a memory subsystem or module 48 and a temperature readout 49; each also being in communication with the first processing means 46.

The application specific subsystem 50 includes power management 52 and RF recovery 54 subsystems, internal and external timing subsystems 56, 58 and at least one, preferably, a plurality of ASIC interfaces 60a, 60b, 60c.

In certain embodiments of the invention, the power management subsystem 52 is adapted to perform at least one of the following functions: (i) convert regulated DC to power the pressure sensor circuitry, (ii) provide protection from excess power input, and (iii) provide orderly powering and hibernation of the pressure sensor circuitry.

In certain embodiments of the invention, the RF recovery subsystem 54 is adapted to perform at least one of the following functions: (i) receive input RF power, (ii) communicate power requirements to external power/communication systems, and (iii) inhibit overload of power circuitry.

In certain embodiments of the invention, the internal and external timing subsystems (or sources) 56, 58 comprise internal and external timing sources and, hence, can be employed to synchronize communication and sensing of the pressure signal.

In certain embodiments of the invention, the ASIC interfaces 60a, 60b, 60c are adapted to receive input from predetermined external controllers, such as SPI, I2C, one-wire or wireless RFID circuitry, and transmit output to same.

As indicated above, in certain embodiments, the pressure sensor communication means includes a wireless communication network or link. In the subject embodiment, the wireless communication network includes antenna means (or an antenna) 22, which is in communication with circuit 21 (see FIG. 1).

In the noted embodiments, the communication means further includes suitable programming and protocols to facilitate wireless communication (or telemetry). As indicated above, in certain embodiments, the pressure sensor 10, i.e. ASIC module, includes the wireless programming and protocols.

Basic pressure sensors, having features that are embodied in or can be readily incorporated into the pressure sensors of the invention, are disclosed in U.S. Pat. No. 6,454,720. The noted sensor features include electronic means associated with the sensor module 30 to provide a measurement signal, communication means for remote transmission of the measurement signal and receipt of control signals, and power supply means. The '720 patent is accordingly incorporated by reference herein in its entirety.

Basic pressure sensor operation and telemetry means are also disclosed in U.S. Pat. Nos. 4,186,079, 5,325,865, 6,113,553, 6,285,899, 6,558,336, 6,731,976 and 6,692,446; each of which is similarly incorporated by reference therein in its entirety.

As indicated above, in certain embodiments of the invention, the pressure sensors of the invention also include at least one system to compensate for variations in temperature and sensor drift. Each of the compensation systems of the invention will now be described in detail.

### Temperature Compensation System

As is well known in the art, the capacitance of a sensing element or member can, and in most instances will, vary with a variation in temperature. A variation in capacitance and, hence, sensor signal represented by the sensing element capacitance will adversely affect the accuracy of the physiological parameter, e.g., intracranial pressure, reflected by the sensor signal.

In certain embodiments of the invention, the pressure sensors thus include a temperature compensation system to correct for reversible temperature effects due to variations in temperature. In the noted embodiments, the temperature compensation system includes at least one temperature sensor. As illustrated in FIG. 3, in certain embodiments, the temperature sensor (or temperature sensing element) 44 is in communication (and cooperates) with the aforementioned ASIC module 40.

According to certain embodiments, temperature induced capacitance variation is characterized by testing the pressure sensing element of the MEMS pressure sensor, e.g., MEMS pressure sensor 32 or a “test” sensing element (which, as discussed in detail below, is manufactured from the same core material) under specified, pre-determined conditions. In certain embodiments, temperature induced capacitance variation is characterized by testing the “test” sensing element (or MEMS sensor formed therewith) under specified time(s) at various pressures.

The temperature induced capacitance variation (or calibrated temperature coefficient) is then stored in the ACIC memory module 48. According to the invention, the temperature induced capacitance variation can be stored in the memory module 48 in any of several formats and/or means, such as two-dimensional tables of capacitance variation versus time, and mathematical functions of capacitance variation as a function of time.

In practice, if a variation in temperature from a predetermined temperature or temperature range, e.g., 30-44° C., is detected by the temperature sensing element or sensor 44, the capacitance variation data stored in the ASIC memory module 48 is employed by the ASIC to correct the measured capacitance and, hence, sensor signal represented by the sensing element capacitance.

### Sensor Drift Compensation System

In certain embodiments of the invention, the pressure sensors include a drift compensation system that is adapted to correct for irreversible drift in sensor components, i.e. pressure sensing elements, due to exposed pressure(s). In certain embodiments, the drift compensation system is also adapted to correct for temperature induced drift.

In certain embodiments, the drift compensation system includes at least one MEMS pressure sensor, which is disposed in the sensor housing, and at least a second test MEMS pressure sensor. In certain embodiments, wherein the drift compensation system is adapted to correct for temperature induced drift, the system includes at least one temperature sensor.

In a preferred embodiment of the invention, the pressure sensing element, i.e. membrane 38, of each MEMS pressure sensor 32 is manufactured from the same lot of material and, hence will have similar chemical and metallurgical properties. More preferably, the membrane (or chip) 38 of each MEMS pressure sensor is acquired (or punched) from adjacent dies on a wafer.

The pressure sensing elements or membranes and, hence, first and second MEMS pressure sensors formed therefrom will thus react under drift inducing stress (e.g., high pressure, high temperature, etc.) in the same manner.

Drift of the MEMS pressure sensors is then characterized by testing the second test MEMS pressure sensor under specified, pre-determined conditions. In certain embodiments, pressure induced drift is characterized by testing the second MEMS pressure sensor under specified time(s) at various pressures. The pressure induced drift characterization is then recorded in a suitable recording medium, e.g., electronic flash memory.

In certain embodiments, temperature induced drift is characterized by testing the second test MEMS pressure sensor under specified time(s) at various temperatures. The temperature induced drift characterization is then also preferably recorded in a suitable recording medium.

According to the invention, the pressure and temperature induced drift characterizations can be recorded in any of several formats and/or means, such as two-dimensional tables of drift versus time, mathematical functions of drift as a function of time, or other parameters, such as fitted data coefficients of mathematical drift functions. The drift characterizations may then accompany all sensors manufactured from the tested material lot or batch, permitting the information to be used in third party applications to compensate for sensor drift (using lot characterization) or individual sensor characterization.

### Telemetric Pressure Sensor Systems

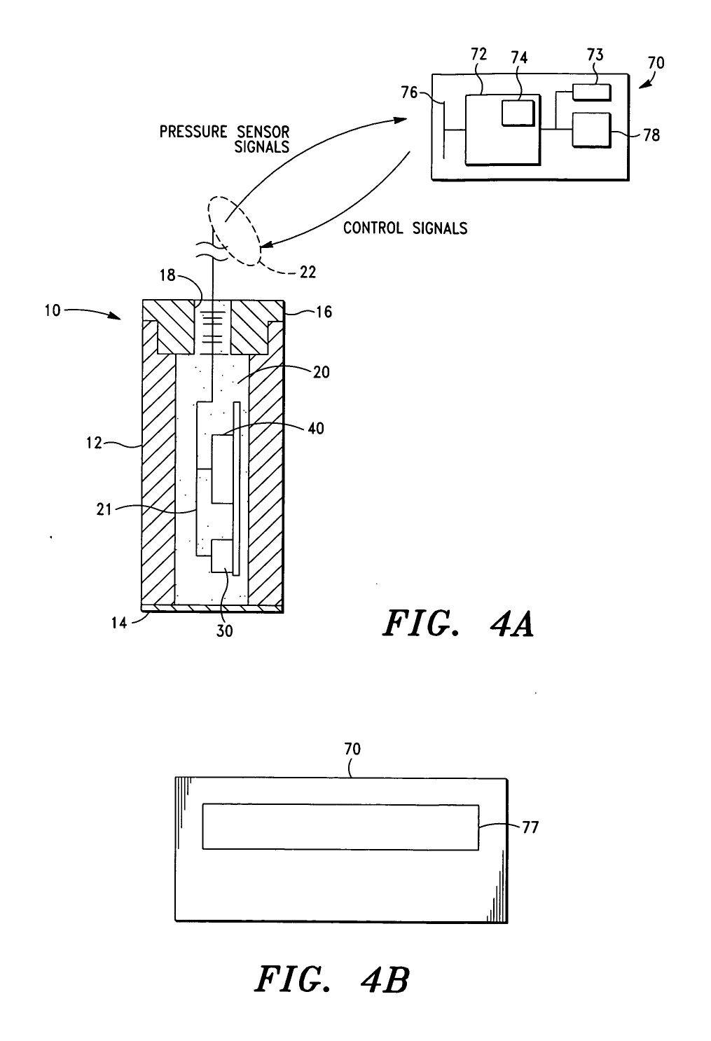

Referring now to FIG. 4A, in certain embodiments, the pressure sensor systems of the invention include the pressure sensor 10 discussed above and an external reader 70. According to the invention, the reader 70 can comprise a stand-alone unit or a hand-held device.

As illustrated in FIG. 4A, the reader 70 includes processing means 72, having a memory module 74 associated therewith, and means for transmitting control signal to and receiving sensor signals from the pressure sensor 10, and a power source 73.

According to the invention, the reader communication means similarly includes a communication network or link. In certain embodiments, the communication network comprises a wireless communication network.

In certain embodiments of the invention, the wireless network includes an antenna 76 or other suitable signal transmission means, which, as illustrated in FIG. 4, is in communication with the reader processing means 72. In the noted embodiments, the processing means 72 includes suitable programming and protocols to facilitate wireless communications.

Referring to FIG. 4B, in certain embodiments of the invention, the reader 70 includes display means 77. In certain embodiments, the display means 77 comprises a visual display.

In the noted embodiments, the reader processing means 72 includes at least one display means module or subsystem, and associated circuitry (i.e. read-out circuitry) that is associated with the reader display means 77.

In certain embodiments, the reader 70 includes audio transmission means. In the noted embodiments, the reader processing means 72 includes at least one audio transmission means module or subsystem, associated circuitry, and a speaker.

In certain embodiments of the invention, the reader further includes [an internal pressure sensor whereby absolute pressure of the environment can be obtained, and from such environmental pressure determine the gauge pressure of the medium surrounding the implanted pressure sensor.

In certain embodiments of the invention, the pressure sensor 10 of the system includes the temperature and drift compensation systems discussed above. In certain embodiments, the pressure induced drift characterization and/or temperature induced drift characterization of the pressure sensor are stored in the reader memory module 74. The drift characterizations can similarly be stored in the memory module 74 in various formats.

In the noted embodiments, the reader processing means 72 is programmed and adapted to correct the pressure sensor output based on the stored pressure and/or temperature induced drift characterizations.

In certain embodiments, the drift compensation system includes at least two MEMS sensors; at least a first MEMS pressure sensor 30 disposed in the sensor housing 12, at least a second MEMS pressure sensor 78 disposed in or associated with the reader 70, and a third test MEMS pressure sensor.

In the noted embodiments, pressure sensing element, i.e. membrane, of each MEMS pressure sensor 30, 78 is similarly preferably punched from adjacent dies on a wafer. The test MEMS pressure sensor is similarly subjected to pressure and/or temperature drift induced characterization testing, and the drift characterizations recorded on a suitable medium and/or stored in the memory module 74.

In an alternative embodiment of the invention, a plurality of pressure sensing elements is employed to construct a plurality of MEMS pressure sensors. Each MEMS pressure sensor is then subjected to pressure and/or temperature drift induced characterization testing. Matching pairs of MEMS pressure sensors, with known, preferably similar drift characteristics, are then disposed in the pressure sensor 10 and reader 70.

In the noted embodiment, the pressure sensing elements can also be punched from adjacent dies on a wafer. Matching pairs of pressure sensing elements and, hence, MEMS pressure sensors formed therefrom, with known, similar drift characteristics, can then be disposed in the pressure sensor 10 and reader 70.

### Pressure Compensation System

In certain embodiments of the invention, the pressure sensor systems include a pressure compensation system to correct for variations in measured internal pressure and atmospheric pressure. The pressure compensation system is further adapted to provide absolute gauge pressure.

In certain embodiments, the pressure compensation system includes a pressure sensing system, which is in communication (and cooperates) with the reader processing means 72. In the noted embodiments, the pressure sensing system similarly includes at least two MEMS pressure sensors; at least a first MEMS pressure sensor 30 disposed in the sensor housing 12, and at least a second (external) MEMS pressure sensor 78 disposed in or associated with the reader 70.

As indicated above, in certain embodiments, the first MEMS pressure sensor 30 is adapted to measure absolute pressure proximate the pressure sensor 10 and, hence, with a cavity, when disposed therein. The second MEMS pressure sensor 78 is adapted to measure absolute atmospheric pressure. In certain embodiments, the measured pressures are stored in the reader memory module 74.

In certain embodiments of the invention, the reader processor means 72 is programmed and adapted to determine gauge pressure as a function of the noted measured absolute pressures. In certain embodiments, the gauge pressure is determined by subtracting the absolute pressure measured by the first MEMS sensor, i.e. pressure proximate the pressure sensor 10, from the absolute atmospheric pressure measured by the second MEMS sensor 78.

### Wired Pressure Sensor Systems

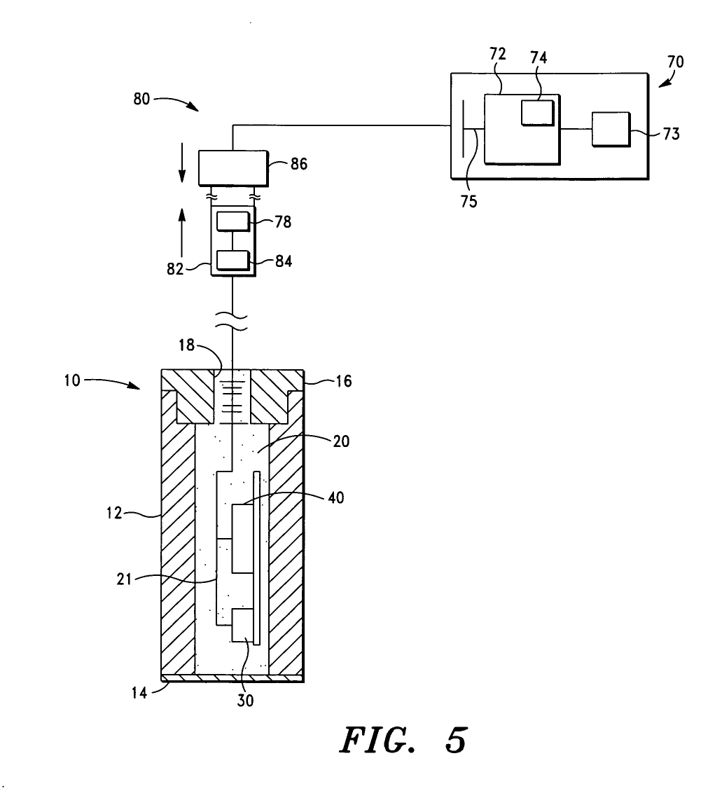

As indicated above, in certain embodiments of the invention, the pressure sensor systems include a wired or direct communication network. Referring now to FIG. 5, there is shown one embodiment of a wired pressure sensor system 80 of the invention.

As illustrated in FIG. 5, the sensor system 80 similarly includes pressure sensor 10 and reader 70. However, in this embodiment, the second MEMS pressure sensor 78 is now disposed in a pressure sensor connector 82, which is in communication with the pressure sensor circuitry 21.

In certain embodiments of the invention, the pressure sensor connector 82 further includes a memory module 84, which is in communication with sensor module 30 and MEMS pressure sensor 78 and adapted to store sensor signals continuously or at predetermined intervals that are transmitted by sensor module 30 and MEMS sensor 78.

As further illustrated in FIG. 4, the reader 70 similarly includes a wired link that is in communication with the reader circuitry 75 and, hence, reader processing means 72. Also associated with the reader wired communication link is a reader connector 86, which is adapted to receive and/or cooperate with the pressure sensor connector 82 to facilitate communication by and between the pressure sensor 10 and reader 70.

### Application Specific Pressure Sensors

Application specific pressure sensor and associated systems of the invention will now be described in detail. However, as indicated above, the pressure sensors and pressure sensor systems of the invention can be employed in various applications, including measuring pressure in a volume (i.e. space, chamber, cavity, substance, tissue, area or the like) in a human body, measuring pressure in a volume in a non-human body, and in non-medical contexts. Thus, although the following discussion generally focuses on measuring pressure in chambers in a human body, the invention is in no way limited to such application.

### Intracranial Pressure (ICP) Sensor

Referring now to FIG. 6, there is shown one embodiment of an ICP sensor 120 of the invention. As illustrated in FIG. 6, the pressure sensor 120 similarly includes a housing or case 122, a membrane 130 disposed at a first end, and a cap 140, having a lumen or feed-through 142 therethrough, disposed on a second end.

The ICP sensor 120 also includes the aforementioned pressure sensor components, modules and subsystems, including the pressure transmitting fluid 20, sensor module or pressure sensing system 30, ASIC module 40, and associated circuitry 21 that facilitates communication by and between the sensor module 30, ASIC module 40 and the communication means, which are disposed within the sensor housing 122.

In certain embodiments of the invention, the membrane 130 similarly comprises a biocompatible material, such as titanium, stainless steel, silicon, glass, or PEEK. In a preferred embodiment, the membrane 14 comprises titanium.

As illustrated in FIG. 6, in certain embodiments, the cap 140 includes an internal region 144 that is adapted to receive and position the pressure sensor antenna 22 therein. In certain embodiments, the cap 140 further includes an engagement region 146 that is adapted to cooperate with the housing 122. In certain embodiments, as illustrated in FIG. 6, the housing 122 includes a recessed region 125 disposed proximate the end of the housing 122 that is opposite the membrane 130.

In the noted embodiments, the recessed region 125 is adapted to securely receive the cap engagement region 146. According to the invention, various conventional engagement means can be employed to facilitate the secure engagement of the cap 140 to the pressure sensor housing 122. In the illustrated embodiment, the cap engagement region 146 includes a raised ring 148 that is configured and positioned to be received by a circular recess 127 in the housing recessed region 125.

In certain embodiments of the invention, the cap 140 comprises a biocompatible polymeric material, such as, without limitation, silicon, nylon, Teflon®, polyvinylchloride, and PEEK. In certain embodiments, the cap 140 comprises a biocompatible metal, such as, without limitation, stainless steel, and titanium. In a preferred embodiment, the cap 140 comprises PEEK and silicone.

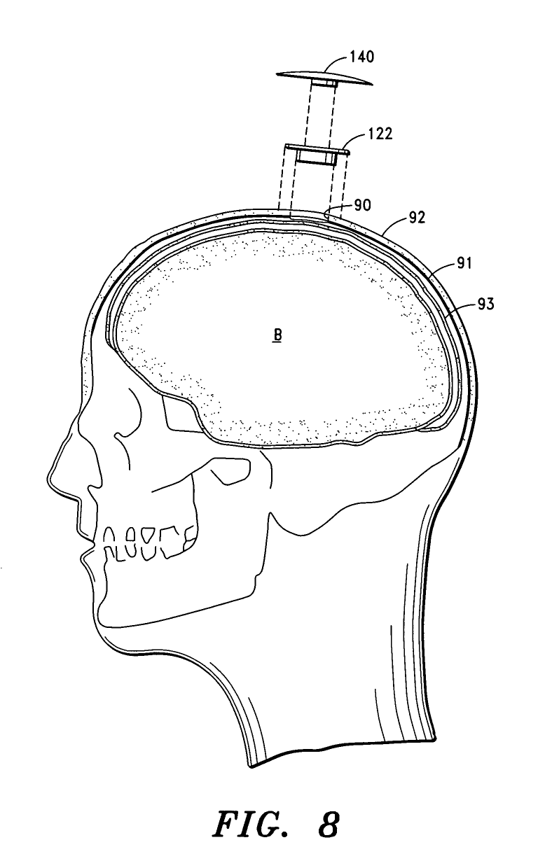

As further illustrated in FIG. 6, to facilitate secure placement in the skull of a subject, in certain embodiments of the invention, the ICP sensor housing 122 includes a plurality of threads (or a threaded portion) 123 that are configured to cooperate with a burr hole in the subject's skull. According to the invention, the sensor housing 122 and threaded portion 123 can comprise various predetermined lengths to accommodate appropriate placement of the distal end 121 of the pressure sensor housing 122 at a desired intracranial position proximate the brain (denoted “B” in FIG. 8), e.g., epidural, subarachnoid or intracerebral location.

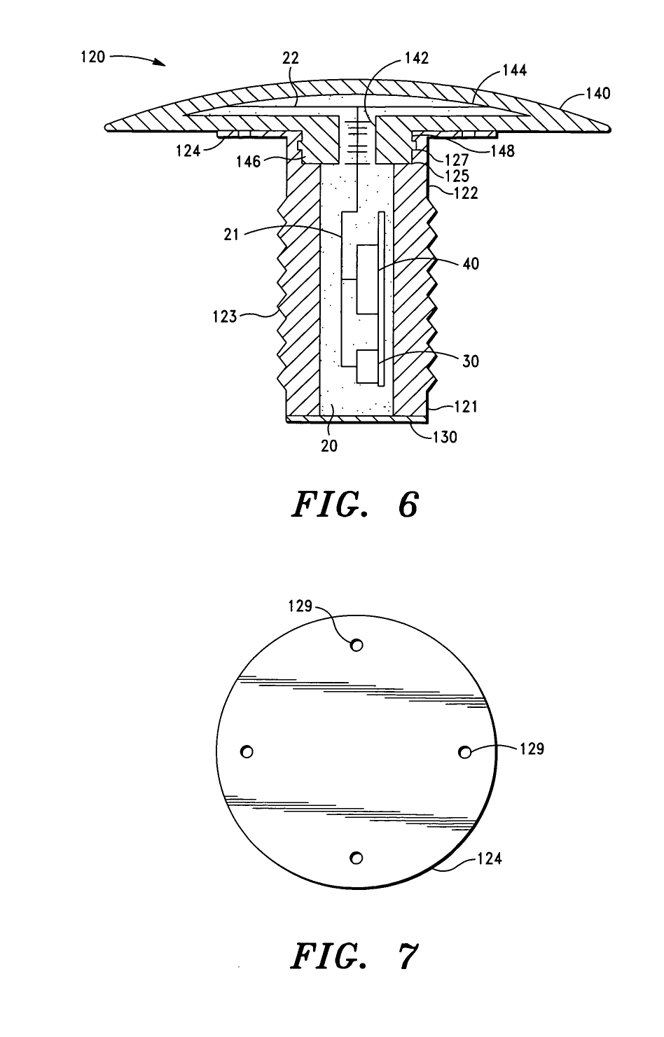

In certain embodiments of the invention, the sensor housing 122 also includes a flanged region 124 that extends substantially perpendicular to the central axis of the sensor housing 122. As illustrated in FIG. 7, in certain embodiments, the flanged region 124 has a substantially circular shape and includes a plurality of spaced holes 129 proximate the edge of the flanged region 124. According to the invention, the holes 129 are configured and positioned to engage cooperating protrusions on an external driving mechanism (not shown) to implant (i.e. screw) the pressure sensor 120 in the skull.

In certain embodiments of the invention, the housing 122 similarly comprises a biocompatible material, such as, without limitation, stainless steel, titanium, silicon, nylon, Teflon®, polyvinylchloride, and PEEK. In a preferred embodiment, the housing 122 comprises titanium.

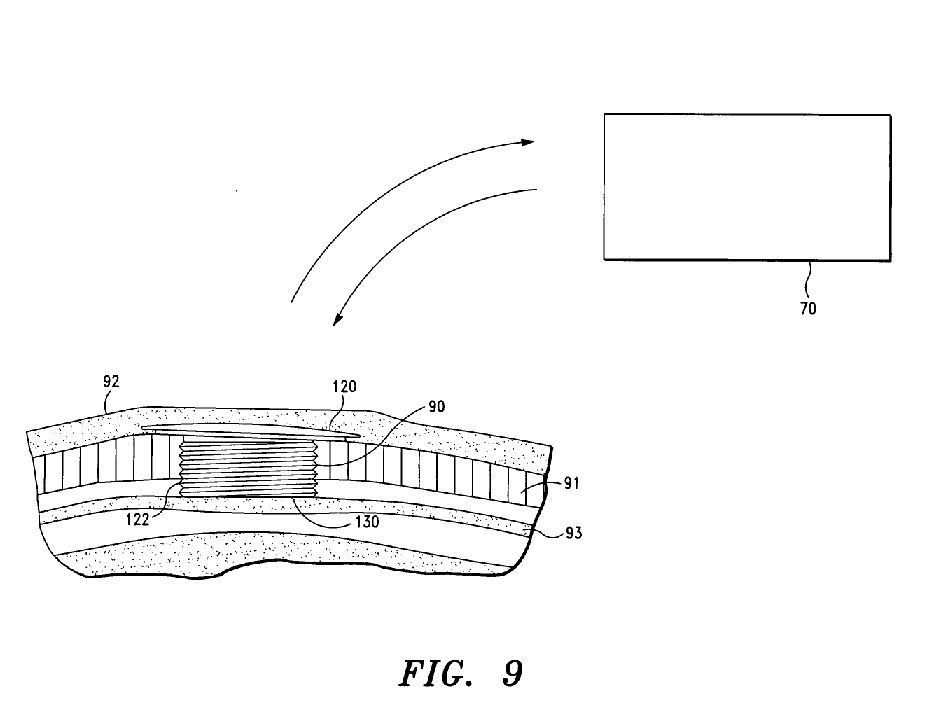

As indicated above, in certain embodiments of the invention, the ICP sensor housing 122 includes a plurality of threads (or a threaded portion) 123 that are configured to cooperate with a burr hole 90 in the subject's skull 91 (see FIGS. 8 and 9). According to the invention, the burr hole 90 (extending through the scalp 92 and skull 91) can be provided by various conventional surgical means.

As illustrated in FIG. 8, once the burr hole 90 is formed, the ICP sensor housing 122 is positioned proximate the burr hole 90. An external driving mechanism is then securely positioned on the housing flange 124, whereby protrusions on the driving mechanism engage the flange holes 129. The driving mechanism is then rotated to screw the sensor housing 122 into and through the burr hole 90.

As indicated above, the sensor housing 122 and threaded portion 123 can comprise various predetermined lengths to accommodate appropriate placement of the distal end 121 of the pressure sensor housing 122 at a desired intracranial position. In certain applications, the sensor housing 122 is threaded into the skull 91 until the sensor membrane 130 contacts the dura matter 93, as shown in FIG. 9.

After placement of the sensor housing 122 in the burr hole 90, the cap 140 is securely positioned on the sensor housing 122.

As illustrated in FIG. 9, the ICP sensor systems of the invention similarly include the aforementioned reader 70. To accommodate signal transmission to and from the reader 70, in the illustrated embodiment, the ICP sensor 120 also includes the aforementioned wireless communication means, including the associated network or link, programming and protocols.

In certain embodiments (not shown), the ICP sensor 120 can include the aforementioned wired communication means.

### Intraocular Pressure Sensor

Before describing the intraocular pressure sensors of the invention, the following brief description of the various anatomical features of the eye is provided to better understand the features and advantages of the invention.

The tear film, which baths the surface of the eye, is about 0.007 mm thick. The tear film has many functions, including hydration, providing nutrients to the epithelial layers, lubrication of the eyelid, and cleaning of the surface of the eye.

The tear film, which baths the surface of the eye, is about 0.007 mm thick. The tear film has many functions, including hydration, providing nutrients to the epithelial layers, lubrication of the eyelid, and cleaning of the surface of the eye.

The cornea, which is the transparent window that covers the front of the eye, is a lens-like structure that provides two-thirds of the focusing power of the eye. The cornea is covered by an epithelium.

The cornea is slightly oval, having an average diameter of about 12 mm horizontally and 11 mm vertically. The central thickness of the cornea is approximately 0.5 mm and approximately 1 mm thick at the periphery.

The sclera is the white region of the eye, i.e. posterior five sixths of the globe. It is the tough, avascular, outer fibrous layer of the eye that forms a protective envelope.

The crystalline lens, which is located between the posterior chamber and the vitreous cavity, separates the anterior and posterior segments of the eye.

The retina is the delicate transparent light sensing inner layer of the eye. The retina faces the vitreous and consists of 2 basic layers: the neural retina and retinal pigment epithelium.

The aqueous humor occupies the anterior chamber of the eye. The aqueous humor provides nutrients to the cornea and lens, and also maintains normal intraocular pressure (IOP).

The limbus is the 1-2 mm transition zone between the cornea and the sclera. This region contains the outflow apparatus of the aqueous humor.

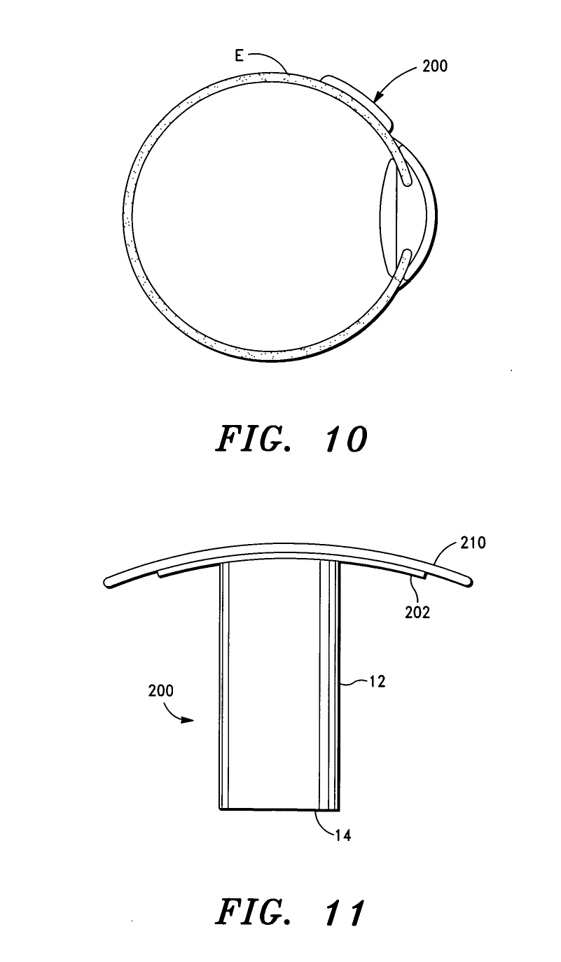

Referring now to FIGS. 10 and 11, one embodiment of an intraocular pressure sensor of the invention, will now be described in detail. The intraocular pressure sensor 200 similarly includes the aforementioned housing or case 12, a membrane 14, and a cap 210 that is adapted to receive the antenna 22.

The intraocular pressure sensor 200 also includes the aforementioned pressure sensor components, modules and subsystems, including the pressure transmitting fluid 20, sensor module or pressure sensing system 30, ASIC module 40, and associated circuitry 21 that facilitates communication by and between the sensor module 30, ASIC module 40 and the communication means, which are disposed within the sensor housing 12.

The sensor housing 12 also includes a flanged region 202 that extends substantially perpendicular to the central axis of the sensor housing 12. However, in the instant embodiment, the flanged region 202 has a curved shape that corresponds to the curvature of the eye (denoted “E” in FIG. 10).

As illustrated in FIG. 11, the cap 210 similarly has a curved shape that preferably corresponds to the shape of the flanged region 202 and curvature of the eye.

The intraocular pressure sensor 200 can be secured at desired locations on the eye by various surgical means. In certain embodiments of the invention, the sensor housing 12 includes engagement means, such as, without limitation, tethers fabricated in the housing for suture attachment.

In certain applications, the intraocular pressure sensor 200 is implanted under the conjunctiva of the subject's eye. In certain applications, the intraocular pressure sensor 200 is attached to the external sclera, whereby the sensor housing 12 extends into the anterior chamber of the eye.

The intraocular pressure sensor systems of the invention similarly include the aforementioned reader 70. To accommodate signal transmission to and from the reader 70, in the illustrated embodiment, the intraocular pressure sensor 200 also includes the aforementioned wireless communication means, including the associated network or link, programming and protocols.

As will readily be appreciated by one having ordinary skill in the art, the pressure sensors, and associated systems and methods of invention provide several significant advantages compared to prior art pressure sensors and methods. Among the advantages are the following:
The provision of pressure sensors, and associated systems and methods, which provide accurate and stable sensor output under varying in vivo and ambient conditions.
The provision of pressure sensors, and associated systems and methods, which provide accurate and stable sensor output over extended periods of time.
The provision of implantable pressure sensors, and associated systems and methods, which provide accurate and stable sensor output under varying in vivo and ambient conditions, and over extended periods of time.
The provision of implantable pressure sensors and associated systems that are suitable for long term implantable use.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. An implantable pressure sensor system, comprising:
a sensor assembly configured and adapted to measure pressure in a volume, said sensor assembly including at least a first MEMS pressure sensor, an application-specific integrated circuit (ASIC) having memory means, a temperature compensation system, a drift compensation system, and power supply means for powering said sensor assembly,
said first MEMS pressure sensor having a first pressure sensing element that is responsive to first exposed pressure, said first pressure sensing element being adapted to generate a first pressure sensor signal representative of said first exposed pressure,
said temperature compensation system being adapted to correct for at least one temperature induced variation in said first pressure sensor signal,
said drift compensation system being adapted to correct for pressure induced first pressure sensing element signal drift.

2. The sensor system of claim 1, wherein said sensor assembly includes a memory module.

3. The sensor system of claim 2, wherein said temperature compensation system includes a first temperature sensor that is responsive to a first temperature, said first temperature sensor being adapted to generate a first temperature signal representative of said first temperature.

4. The sensor system of claim 3, wherein said first temperature sensor is disposed proximate said first MEMS pressure sensor.

5. The sensor system of claim 3, wherein said temperature compensation system further includes at least one calibrated temperature coefficient, said calibrated temperature coefficient being stored in said ASIC memory means.

6. The sensor system of claim 3, wherein said ASIC is adapted to correct for said temperature induced variation in said first pressure signal as a function of said first temperature signal and said calibrated temperature coefficient.

7. The sensor system of claim 2, wherein said drift compensation system includes at least one pressure induced drift characterization.

8. The sensor system of claim 7, wherein said drift compensation system is further adapted to correct for temperature induced first pressure sensing element signal drift.

9. The sensor system of claim 8, wherein said drift compensation system includes at least one temperature induced drift characterization.

10. The sensor system of claim 9, wherein said drift compensation system includes memory means adapted to store said pressure and temperature induced drift characterizations and at least one algorithm adapted to correct said first capacitance variation output from said first sensing element with said pressure and temperature induced drift characterizations.

11. The sensor system of claim 1, wherein said sensor assembly includes a sensor housing having an internal chamber.

12. The sensor assembly of claim 11, wherein said first MEMS pressure sensor is disposed in said sensor housing internal chamber.

13. The sensor system of claim 12, wherein said sensor assembly includes a pressure compensation system adapted to correct for variations in measured pressures proximate said first MEMS pressure sensor and atmospheric pressure.

14. The sensor system of claim 13, wherein said pressure compensation system includes a second MEMS sensor having a second pressure sensing element that is responsive to second exposed pressure, said second pressure sensing element being adapted to generate a second pressure sensor signal representative of said second exposed pressure.

15. The sensor system of claim 14, wherein said second MEMS pressure sensor is disposed external of said sensor housing and wherein said second exposed pressure comprises atmospheric temperature.

16. The sensor system of claim 14, wherein said first and second pressure sensing elements are formed from adjacent dies on a wafer.

17. The sensor system of claim 15, wherein said pressure compensation system includes an external reader in communication with said first and second MEMS pressure sensors, said reader including processing means adapted to receive said first and second pressure signals and determine gauge pressure therefrom.

18. The sensor system of claim 14, wherein said first and second MEMS pressure sensors have an accuracy of at least approximately +/−0.75 mmHg on a scale of approximately 500-1000 mmHg.

19. The sensor system of claim 1, wherein said sensor assembly comprises a physiologic sensor.

20. The sensor system of claim 19, wherein said sensor assembly includes at least one additional physiological sensor.

21. The sensor system of claim 20, wherein said physiological sensor comprises a sensor selected from the group consisting of a pO₂ sensor, pCO₂ sensor and SpO₂ sensor.

22. The sensor system of claim 1, wherein said sensor assembly includes a communication network adapted to facilitate communication by and between said sensor assembly and an external monitor.

23. The sensor system of claim 1, wherein said communication network comprises a wireless network.

24. The sensor system of claim 1, wherein said communication network comprises a wired network.

25. An implantable pressure sensor system, comprising:
a sensor assembly configured and adapted to measure pressure in a volume, said sensor assembly including at least one MEMS pressure sensor, a temperature compensation system, a drift compensation system, and a pressure compensation system,
said MEMS pressure sensor being responsive to exposed pressure and adapted to generate a pressure sensor signal representative of said exposed pressure,
said temperature compensation system being adapted to correct for temperature induced variations in said pressure sensor signal,
said drift compensation system being adapted to correct for pressure and temperature induced drift of said pressure sensor signal,
said pressure compensation system being adapted to correct for variations in measured pressures of said MEMS pressure sensor and atmospheric pressure.

26. An implantable pressure sensor system, comprising:
a sensor assembly configured and adapted to measure pressure in a volume, said sensor assembly including a MEMS pressure sensor, an application-specific integrated circuit (ASIC), a temperature compensation system, a drift compensation system, and a pressure compensation system,
said MEMS pressure sensor having a pressure sensing element that is adapted to generate a capacitance variation signal in response to exposed pressure,
said ASIC being adapted to generate a pressure signal with said capacitance variation signal, said pressure signal being representative of said exposed pressure,
said temperature compensation system being adapted to correct for temperature induced variations in said pressure signal,
said drift compensation system being adapted to correct for pressure and temperature induced drift of said capacitance variation signal,
said pressure compensation system being adapted to correct for variations in measured pressures of said MEMS pressure sensor and atmospheric pressure.

27. An implantable pressure sensor system, comprising:
a sensor assembly configured and adapted to measure pressure in a volume, said sensor assembly including a MEMS pressure sensor, a digital capacitance system, a temperature compensation system, a drift compensation system, and a pressure compensation system,
said MEMS pressure sensor being adapted to generate a capacitance signal in response to exposed pressure,
said digital capacitance system being adapted to convert said capacitance signal to a pressure signal, said pressure signal being representative of said exposed pressure,
said temperature compensation system being adapted to correct for temperature induced variations in said capacitance signal,
said drift compensation system being adapted to correct for pressure and temperature induced drift of said capacitance signal,
said pressure compensation system being adapted to correct for variations in measured pressures of said MEMS pressure sensor and atmospheric pressure.

28. A method for measuring pressure in a chamber of a human body, comprising the steps of:
providing a sensor assembly having a MEMS pressure sensor, an application-specific integrated circuit (ASIC), a temperature compensation system, a drift compensation system, a pressure compensation system and power supply means for powering said sensor assembly, said MEMS pressure sensor being adapted to generate a capacitance variation signal in response to exposed pressure, said ASIC being adapted to generate a pressure signal with said capacitance variation signal, said pressure signal being representative of said exposed pressure, said temperature compensation system being adapted to correct for temperature induced variations in said capacitance variation signal, said drift compensation system being adapted to correct for pressure and temperature induced drift of said capacitance variation signal, said pressure compensation system being adapted to correct for variations in measured pressures of said MEMS pressure sensor and atmospheric pressure;
disposing said sensor assembly in a chamber of a human body; and
measuring pressure in said chamber with said sensor assembly, whereby a first pressure signal representative of said chamber pressure is generated.

29. The method of claim 28, wherein said chamber comprises an anterior chamber of an eye.

30. The method of claim 28, wherein said chamber comprises an intracranial chamber.

31. The method of claim 28, wherein said sensor assembly includes wireless communication means for wirelessly transmitting said first pressure signal to a remote receiving apparatus.
